(19) United States
(12) Patent Application Publication
(10) Pub. No.: US 2006/0128004 A1
(43) Pub. Date: Jun. 15, 2006

(54) Process and an apparatus for producing calcium carbonate via an enzymatic pathway

(76) Inventors: Frederick Anctil, St. Albert (CA); Roger Gaudreault, Kingsey Falls (CA)

Correspondence Address: WARE FRESSOLA VAN DER SLUYS &;ADOLPHSON, LLP, BRADFORD GREEN BUILDING 5, 755 MAIN STREET, P O BOX 224, MONROE, CT, 06468, US

(21) Appl. No.: 10/521,395

(22) PCT Filed: Jul. 16, 2003
(86) PCT No.: PCT/CA03/01070

§ 371 Date: Aug. 15, 2005

(30) Foreign Application Priority Data
Jul. 16, 2002 (CA) 2394073

(51) Int. Cl. A61L 9/01; C12M 1/00

(52) U.S. Cl. 435/266; 435/289.1; 435/299.1

(57) ABSTRACT

The invention relates to a method for improving the production yield of precipitated calcium carbonate (PCC). The improvement resides in the use of an enzymatic bioreactor that supplies HCO₃ into the reaction enabling to produce PCC without bubbling gaseous carbon dioxide in the aqueous solution. The present invention has an industrial applicability, namely in the pulp and paper industry.

### FIELD OF THE INVENTION

The present invention generally relates to the field of calcium carbonate production obtained by precipitation. More particularly, it concerns a process for preparing precipitated calcium carbonate (hereinafter referred to as PCC) involving the use of an enzyme that directly supplies a source of bicarbonate ions which are required by the PCC production process. The present invention is particularly useful in the pulp and paper industry.

### BACKGROUND OF THE INVENTION

Production of CaCO₃

Calcium carbonate is used in different fields, most importantly in the pulp and paper industry, where it is largely used for coating the paper after it has been pressed, thereby increasing the printing quality of the paper, as well as its color, smoothness, and strength.

The traditional process of forming solid CaCO₃ consists in bubbling carbon dioxide in milk of lime (Ca(OH)₂). This process allows a sufficient amount of CO₂ to solubilize and produce solid calcium carbonate according to the following equation:
Ca(OH)₂+CO₂<-------->CaCO₃+H₂O [1]

This reaction is a rather slow reaction and one drawback thus encountered with the same is its low production yield as compared to the need of CaCO₃ in the pulp and paper industry or in other fields.

There is thus presently a need for a process for the production of CaCO₃ that provides a better production yield.

Conversion of CO₂ into Hydrogen Ions and Bicarbonate Ions

EP0991462; AU7753398; WO9855210; CA2291785 in the name of the Applicant disclose the use of the enzyme carbonic anhydrase to catalyse the hydration of CO₂ into hydrogen ions and bicarbonate ions. The reaction that allows for the production of bicarbonate in the presence of the enzyme is represented by equation [2]: 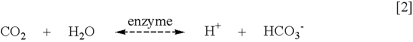
The hydration kinetics of CO₂, with or without enzyme, has been the object of several scientific works. The reaction rate constant of non-catalysed (without enzyme) reactions is in the order of 0,035s⁻¹ (25° C., pH=7)¹. At an elevated pH (pH>10), the hydroxyl ions (OH⁻) contribute to the catalysis, and consequently, the reaction rate constant can attain 8.5×10³ s⁻¹ at 25° C.². The catalysed reaction (with enzyme), can also be associated with an elevated reaction rate constant in the order of 1,4×10⁶ (25° C., pH=8.8)³. The ratio of the two reaction rate constants allows one to predict the CO₂hydration kinetics, which can be in the order of 50 million times more elevated when an enzyme is present and depending on the experimental conditions.
1 (Carbonic Anhydrase: Zinc and the Mechanisms of Catalysis. Biology and Chemistry of the Carbonic Anhydrolases; Annals of the New-York Academy of Sciences, 429 (1984) pp.26 to 48, (Coleman J. E.); “The Carbonic Dioxide Hydration activity of carbonic anhydrase I. Stop-Flow Kinetic studies on the Native Human Isoenzymes B and C”, Journal of Biological Chemistry, 246(8) (1971), pp. 2561 to 2573 (Khalifah, R. G.); Biochimie (2000) DeBoeck Université (Garett et al.) 2 ((Carbonic Anhydrase: Zinc and the Mechanisms of Catalysis. Biology and Chemistry of the Carbonic Anhydrolases; Annals of the New-York Academy of Sciences, 429 (1984) pp.26 to 48, (Coleman J. E.); Carbonic Anhydrase Kinetics and Molecular Function: The carbonic Anhydrase, Plenum Press, pp. 49 to 69 (1991), Khalifah et al.) 3 (“The Carbonic Dioxide Hydration activity of carbonic anhydrase I. Stop-Flow Kinetic studies on the Native Human Isoenzymes B and C”, Journal of Biological Chemistry, 246(8) (1971), pp. 2561 to 2573 (Khalifah, R. G. )

### SUMMARY OF THE INVENTION

An object of the present invention is to provide a process and an apparatus for the production of CaCO₃ that satisfies the above-mentioned need for a process that provides an increased production yield of precipitated calcium carbonate (PCC). In accordance with the present invention, that object is achieved with a process for producing CaCO₃, charaterized in that it comprises the steps of:
a) catalysing the hydration of CO₂contained in a CO₂-containing gas by means of an enzyme capable of catalysing the hydration of dissolved CO₂into hydrogen ions and bicarbonate ions, thereby producing a solution containing bicarbonate ions and hydrogen ions;
b) reacting the bicarbonate ions contained in the solution obtained in step a) with calcium ions, thereby producing a solution containing CaCO₃; and
c) precipitating the CaCO₃ contained in the solution obtained in step b).

The enzyme used in the process is preferably carbonic anhydrase or an analogue thereof.

In accordance with a preferred aspect, step b) comprises the step of mixing the solution obtained in step a) with Ca(OH)₂, thereby providing the calcium ions. In that case, the CaCO₃ is thus produced from milk of lime (Ca(OH)₂) and CO₂as in the prior art, although it shows an improved yield, as explained hereinafter. As mentioned above, the traditional process of forming CaCO₃ consists in bubbling carbon dioxide in milk of lime (Ca(OH)₂). This process produces solid calcium carbonate according to the following equation:
Ca(OH)₂+CO₂<-------->CaCO₃+H₂O [1]

This non catalyzed solubilization of gaseous CO₂in an aqueous medium implies the following series of natural reversible reactions. The equilibrium constants of these natural reactions are well known in the prior art.
CO₂+H₂O<-->H₂CO₃ K≅600 à25° C. (K≡[CO₂]/[H₂CO₃]) [3]
H₂CO₃<->H⁺+HCO₃⁻ K=4,47×10⁻⁷ à25° C. (K=[HCO₃⁻][H⁺]/[H₂CO₃]) [4]
HCO₃⁻<-->H⁺+CO₃²⁻ K=4,70×10⁻¹¹à25° C. (K=[CO₃²⁻][H⁺]/[HCO3⁻]) [5]

From a thermodynamic point of view, the large equilibrium constant associated to equation [3] translates into a weak inherent tendency to produce the acid (K_{eq}=[CO₂]/[H₂CO₃]≅600) (Chemistry of the Elements (₂nd edition, Butterworth Heinemann, p. 310 (1997)).

Equation [3], representing the formation of carbonic acid (H₂CO₃), is in reality the limiting step of the process for producing bicarbonate.

Thus, the use of an enzyme, preferably carbonic anhydrase, which is specific to CO₂molecules, avoids the step of carbonic acid formation. In other words, the enzyme catalyses the hydration of CO₂by eliminating the limiting step of the process. As mentioned above, the reaction that allows for the production of bicarbonate in the presence of the enzyme is represented by equation [2]: 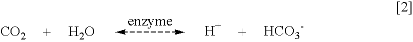
Reaction [2] allows for the production of an aqueous solution having a high HCO₃⁻ ion content that is used in the process of producing PPC. This concept is represented in the following equation:
HCO₃⁻H⁺+Ca(OH)₂<-->CaCO₃+2H₂O [6]

From equations [1] and [6], one can see that the reaction for producing PCC performs better in the presence of an enzymatic bioreactor supplied with gaseous CO₂which in turn directly supplies HCO₃⁻ to the process for preparing precipitated calcium carbonate. The improvement resides not only in the increased hydration rate of CO₂, but also in the increased amounts of CaCO₃ formed in a given reaction time. The variation of Gibbs free energy (ΔG) for each of the reactions will indeed allow one to determine which of the two chemical reactions is favourable from a thermodynamic point of view.

Reaction [6], having a ΔG in the order of −119 kJ/mol, is more favourable from a thermodynamic point of view than reaction [1] which has a ΔG value in the order of −74 kJ/mol. The respective equilibrium constant of these two chemical reactions is calculated using equation [7]:
−ΔG=RT In (K_{eq}) [7]

The equilibrium constant of a chemical reaction not containing enzyme is about 9,36×10¹² at 298K, while a catalysed reaction making use of an enzyme is about 7,24×10²⁰. While the equilibrium constant is higher for a reaction containing an enzyme, the chemical equilibrium is more favourable towards the product (CaCO₃) then to the reagents, and consequently, a better yield is reached.

In accordance with a preferred aspect of the invention, the step a) of CO₂hydration is performed in a bioreactor comprising a reaction chamber filled with the enzyme and step b) is performed in at least one separate reaction tank, the process further comprising a step of directing a flow of the solution from the bioreactor into the reaction tank.

Also preferably, step c) of precipitating is performed in the reaction tank. In the present context, precipitating the CaCO₃ means the formation of separable solid CaCO₃ from the solution or crystallization of the CaCO₃.

### BRIEF DESCRIPTION OF THE FIGURES

FIG. 1 is a schematic representation of a first installation suitable for producing precipitated calcium carbonate according to the process of the present invention.

FIG. 2 is a schematic representation of a second installation suitable for producing precipitated calcium carbonate according to the process of the present invention.

### DESCRIPTION OF PREFERRED EMBODIMENTS OF THE INVENTION

The invention consists in replacing the step of bubbling gaseous CO₂in the milk of lime by a direct input of HCO₃⁻ in an aqueous solution so as to accelerate the production rate of CaCO₃.

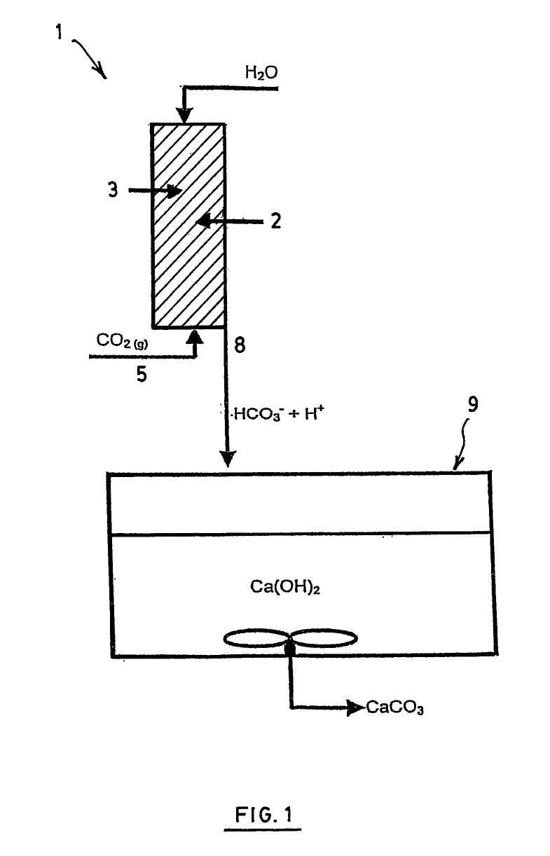

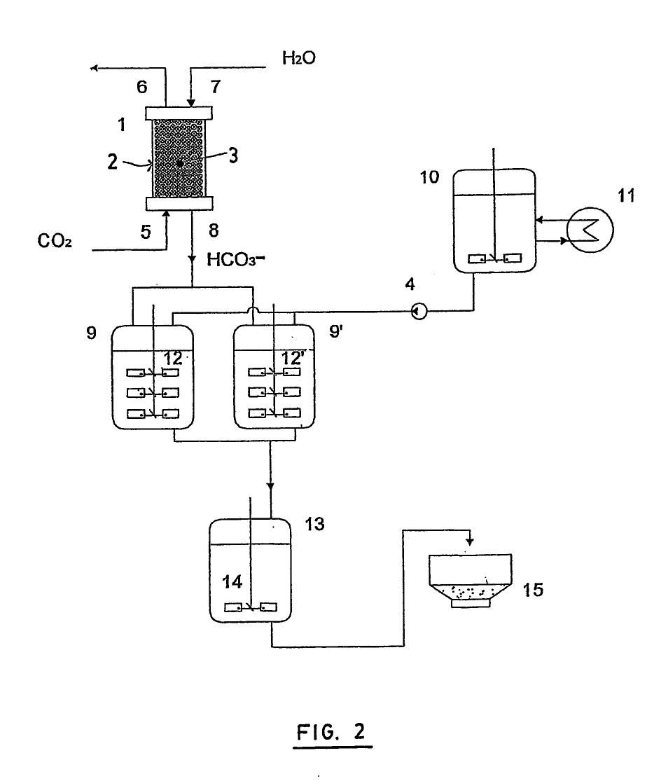

Referring to FIGS. 1 or 2, a CO₂conversion bioreactor (1) having a reaction chamber (2) filled with carbonic anhydrase is used. Preferably, the reaction chamber (2) is filled with packing (3) on which carbonic anhydrase is immobilized.

The most common form which the CO₂conversion bioreactor (1) can take is the one containing a packed column where gaseous CO₂or other effluents containing CO₂penetrate towards the bottom of column (5) at a high concentration (>10 000 ppm) and exit by the outlet (6) at a concentration in the order of 500 ppm. As for example, a bioreactor as the one described in WO9855210; CA2291785 is suitable for performing the process. The dimensions of the column depend on both the ratio of gas vs. liquid flow rate and the difference of concentrations between the inlet (5) and the outlet (6). The liquid phase, containing water and a buffer solution so as to allow the pH of the enzymatic medium to be maintained, flows into the bioreactor by an upper inlet (7) and crosses the gas flow while flowing towards the bottom of the reaction chamber where the outlet (8) is located. During the crossing, water, CO₂, and the enzyme react according to equation [2]. The concentration of gaseous CO₂then decreases since water fills with HCO₃⁻ and H⁺ ions in equivalent proportions upon transformation of the CO₂into HCO₃⁻ and H⁺ ions. The gas concentration at the outlet (6) is an indicator of the amount of HCO₃⁻ dissolved. However, it is preferable to measure the amount of dissolved ions in the solvent (8) found in the pipes.

The solution containing the ions is then directed towards one or more reaction tanks (9 or 9′) that contain the milk of lime. It is worth mentioning that the milk of lime is preferably kept at an optimal concentration so as to produce homogenous PCC crystals. Turning now to FIG. 2, the reaction tanks (9 and 9′) are used in parallel and can be numerous. The temperature at which milk of lime is kept can be adjusted in the conditioning tank (10) by means of heating/cooling system (11). This system allows the solution to be either heated or cooled depending on the HCO₃⁻ solution properties. Afterwards, an exact amount of milk of lime is added into the reaction tanks (9 and 9′) by means of a volumetric pump (4). Indeed, when the reagents are placed together, CaCO₃ crystals are formed within the first moments of contact. The content of the reaction tank (9 and 9′) is preferably stirred by means of conventional mixer (12 or 12′) to prevent the CaCO₃ crystals from settling.

Finally, the content of each of the reaction tanks (9 and 9′) is alternatively transferred one after another into a buffer tank (13) where the liquid and the calcium carbonate solution await to enter the filtration system (15) where they are to be separated from one another. A mixing system (14) can be used to avoid the formation of deposits (sedimentation) along the bottom of the buffer tank (13). The use of a buffer tank (13) enables the reaction tanks (9 and 9′) to remain available to receive solutions so as to keep the process continuous.

It is worth mentioning that it is also possible to directly add the enzyme in free form into the reaction chamber (9 or 9′). However, in doing so, it can compromise the precipitate purity as well as consuming large amounts of enzyme. Indeed, immobilization of anhydrase on the packing (3) allows the enzyme to be reused many times over until its performance is no longer optimal.

The process and the apparatus according to the invention are advantageously applicable in the industry of pulp and paper for improving the production yield of CaCO₃ used for coating the paper.

It is however worth mentioning that the invention could also be advantageous in other fields requiring the use of CaCO₃.

Although preferred embodiments of the present invention have been described in detail herein and illustrated in the accompanying drawings, it is to be understood that the invention is not limited to these precise embodiments and that various changes and modifications may be effected therein without departing from the scope or spirit of the present invention.

1. A process for producing CaCO₃, charaterized in that it comprises the steps of:
a) catalysing the hydration of CO₂contained in a CO₂-containing gas by means of an enzyme capable of catalysing the hydration of dissolved CO₂into hydrogen ions and bicarbonate ions, thereby producing a solution containing bicarbonate ions and hydrogen ions;
b) reacting the bicarbonate ions contained in the solution obtained in step a) with calcium ions, thereby producing a solution containing CaCO₃; and
c) precipitating the CaCO₃ contained in the solution obtained in step b).

2. A process according to claim 1, characterized in that step b) comprises the step of:
mixing the solution obtained in step a) with Ca(OH)₂, thereby providing said calcium ions.

3. A process according to claim 1 or 2, characterized in that said enzyme is carbonic anhydrase or an analogue thereof.

4. A process according to any one of claims 1 to 3, characterized in that step a) of hydration of CO₂is performed in a bioreactor comprising a reaction chamber filled with said enzyme and the step b) is performed in at least one separate reaction tank, the process further comprising a step of directing a flow of said solution from said bioreactor into said reaction tank.

5. A process according to claim 4, characterized in that the reaction chamber is filled with packing on which the enzyme is immobilized.

6. A process according to any one of claims 1 to 5, characterized in that step b) is performed under stirring to prevent the calcium carbonate from settling.

7. A process according to any one of claims 1 to 6, characterized in that it comprises an additional step of:
d) separating the precipitate of CaCO₃ of step c) from the solution.

8. A process according to claim 6, characterized in that step d) of separating consists of filtering.

9. An apparatus for producing CaCO₃ according to the process defined in claim 1, characterized in that it comprises:
catalyzing means for catalysing the hydration of the CO₂into bicarbonate ions and hydrogen ions;
reacting means for reacting the bicarbonate ions obtained in the catalyzing means with calcium ions to produce CaCO₃; and
precipitating means for precipitating the CaCO₃ obtained in the reacting means.

10. An apparatus according to claim 9, characterized in that the means for catalyzing the hydration of the CO₂comprises a bioreactor comprising:
a gas inlet for receiving gaseous CO₂;
a liquid inlet for receiving an aqueous liquid;
a reaction chamber in fluid communication with the gas inlet and the liquid inlet, the reaction chamber containing therein enzymes capable of catalysing the hydration of dissolved CO₂into bicarbonate ions and hydrogen ions; and
a liquid outlet in fluid communication with the reaction chamber for discharging a solution of bicarbonate ions and hydrogen ions.

11. An apparatus as claimed in claim 10, characterized in that the enzyme is carbonic anhydrase.

12. An apparatus as claimed in claim 10 or 11, characterized in that the means for reacting the bicarbonate ions with calcium ions and precipitating CaCO₃ is at least one reaction tank having an inlet in fluid communication with the liquid outlet of the bioreactor and an outlet to discharge a solution containing CaCO₃.

13. An apparatus as claimed in claim 12, characterized in that it comprises a buffer tank having an inlet in fluid communication with the outlet of the at least one reaction tank for receiving and reserving the solution obtained in said at least one reaction tank for a further treatment.

14. An apparatus according to claim 13, characterized in that it comprises a filter in fluid communication with said buffer tank to separate the CaCO₃ from the solution.
